(19) United States
(12) Patent Application Publication
(10) Pub. No.: US 2024/0055022 A1
(43) Pub. Date: Feb. 15, 2024

(54) NONLINEAR LIGHT ABSORPTION MATERIAL, RECORDING MEDIUM, METHOD FOR RECORDING INFORMATION, AND METHOD FOR READING INFORMATION

(71) Applicant: Panasonic Intellectual Property Management Co., Ltd., Osaka (JP)

(72) Inventors: MASAKO YOKOYAMA, Osaka (JP); TERUHIKO SAITO, Osaka (JP); KOTA ANDO, Osaka (JP); HIDEKAZU ARASE, Hyogo (JP)

(21) Appl. No.: 18/489,053

(22) Filed: Oct. 18, 2023

Related U.S. Application Data
(63) Continuation of application No. PCT/JP2022/012115, filed on Mar. 17, 2022.

(30) Foreign Application Priority Data
May 20, 2021 (JP) 2021-085356

(51) Int. Cl. G11B 7/244; C07C 15/50; C07C 25/24; C07C 22/08; C07C 39/21; C07C 63/66; C07C 69/76; C07C 49/796; C07C 233/65; C07C 255/50; C07C 47/548; C07C 321/26; C07C 309/32; C07C 327/26; C07C 317/14; C07C 311/16; C07C 211/45; C07C 205/06

(52) U.S. Cl. CPC G11B 7/244; C07C 15/50; C07C 25/24; C07C 22/08; C07C 39/21; C07C 63/66; C07C 69/76; C07C 49/796; C07C 233/65; C07C 255/50; C07C 47/548; C07C 321/26; C07C 309/32; C07C 327/26; C07C 317/14; C07C 311/16; C07C 211/45; C07C 205/06

(57) ABSTRACT

A nonlinear light absorption material includes a compound represented by the following formula (1) as a main component:

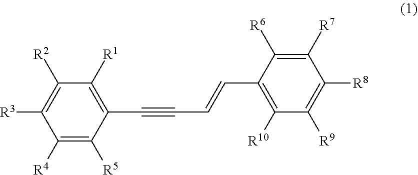

in the formula (1), R¹ to R¹⁰ are each independently a hydrogen atom, a halogen atom, a saturated hydrocarbon group, a halogenated alkyl group, an unsaturated hydrocarbon group, a hydroxyl group, a carboxyl group, an alkoxycarbonyl group, an aldehyde group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

### BACKGROUND

### 1. Technical Field

The present disclosure relates to a nonlinear light absorption material, a recording medium, a method for recording information, and a method for reading information.

### 2. Description of the Related Art

Among optical materials such as light absorption materials, a material having a nonlinear optical effect is called a nonlinear optical material. The nonlinear optical effect means that when a material is irradiated with strong light such as laser light, an optical phenomenon proportional to the square or higher power of the electric field of the irradiation light occurs in the material. Examples of the optical phenomenon include absorption, reflection, scattering, and emission. Examples of the quadratic nonlinear optical effect proportional to the square of the electric field of the irradiation light include second harmonic generation (SHG), a Pockels effect, and a parametric effect. Examples of the tertiary nonlinear optical effect proportional to the cube of the electric field of the irradiation light include two-photon absorption, multi-photon absorption, third harmonic generation (THG), and a Kerr effect. In the present specification, multi-photon absorption such as two-photon absorption may be called nonlinear light absorption. A material that can perform nonlinear light absorption may be called a nonlinear light absorption material. In particular, a material that can perform two-photon absorption may be called a two-photon absorption material.

Many studies have been actively conducted on nonlinear optical materials. In particular, as nonlinear optical materials, inorganic materials from which single crystals can be easily prepared have been developed. In recent years, nonlinear optical materials made of organic materials are expected to be developed. Organic materials, compared to inorganic materials, have not only a high degree of design freedom but also a large nonlinear optical constant. Furthermore, in organic materials, a nonlinear response is performed at a high speed. In the present specification, a nonlinear optical material including an organic material may be called an organic nonlinear optical material.

### SUMMARY

In one general aspect, the techniques disclosed here feature a nonlinear light absorption material comprising a compound represented by the following formula (1) as a main component:

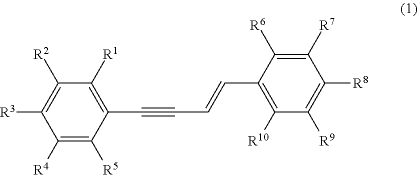

in the formula (1), R¹ to R¹⁰ are each independently a hydrogen atom, a halogen atom, a saturated hydrocarbon group, a halogenated alkyl group, an unsaturated hydrocarbon group, a hydroxyl group, a carboxyl group, an alkoxycarbonyl group, an aldehyde group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a flow chart of a method for recording information using a recording medium including a nonlinear light absorption material according to an embodiment of the present disclosure;

FIG. 1B is a flow chart of a method for reading information using a recording medium including a nonlinear light absorption material according to an embodiment of the present disclosure;

FIG. 2A is a graph showing the ¹H-NMR spectrum of compound (2)-1; and

FIG. 2B is a graph showing the ¹³C-NMR spectrum of compound (2)-1.

### DETAILED DESCRIPTIONS

### Underlying Knowledge Forming Basis of the Present Disclosure

As organic nonlinear optical materials, two-photon absorption materials have received particular attention. The two-photon absorption means a phenomenon that a compound almost simultaneously absorbs two photons and transits to an excited state. As the two-photon absorption, simultaneous two-photon absorption and stepwise two-photon absorption are known. The simultaneous two-photon absorption may be also called non-resonance two-photon absorption. The simultaneous two-photon absorption means two-photon absorption in a wavelength region where there is not one-photon absorption band. The stepwise two-photon absorption may also be called resonance two-photon absorption. In the stepwise two-photon absorption, a compound absorbs a first photon and then further absorbs a second photon to transit to a higher excited state. In the stepwise two-photon absorption, the compound absorbs two photons sequentially.

In the simultaneous two-photon absorption, the amount of light absorbed by a compound is generally proportional to the square of the strength of the irradiation light and exhibits nonlinearity. The amount of light absorbed by a compound can be used as an index of the efficiency of two-photon absorption. When the amount of light absorbed by a compound exhibits nonlinearity, for example, it is possible to cause absorption of light by the compound at only near the focal point of laser light having a high electric field strength. That is, in a sample including a two-photon absorption material, the compound can be excited at a desired position only. Thus, compounds causing simultaneous two-photon absorption induce a significantly high spatial resolution and are therefore being studied for applications such as a recording layer of a three-dimensional optical memory or a photocurable resin composition for three-dimensional (3d) laser microfabrication.

In two-photon absorption materials, a two-photon absorption cross-section (GM value) is used as an index indicating the efficiency of two-photon absorption. The unit of the two-photon absorption cross-section is GM (10⁻⁵⁰ cm⁴·s·molecule⁻¹·photon⁻¹). A large number of organic two-photon absorption materials having a large two-photon absorption cross-section have been proposed. For example, many compounds having a large two-photon absorption cross-section exceeding 500 GM have been reported (for example, Harry L. Anderson et al, “Two-Photon Absorption and the Design of Two-Photon Dyes”, Angew. Chem. Int., Ed. 2009, Vol. 48, pp. 3244-3266). However, in most of the reports, the two-photon absorption cross-sections have been measured using laser light having a wavelength of longer than 600 nm. In particular, near infrared rays having a wavelength longer than 750 nm may also be used as the laser light.

However, in order to apply the two-photon absorption material to an industrial use, a material expressing a two-photon absorption property when irradiated with laser light having a shorter wavelength is required. For example, in the field of three-dimensional optical memory, laser light having a short wavelength can realize a finer condensed light spot and therefore can improve the recording density of a three-dimensional optical memory. In also the field of three-dimensional (3d) laser microfabrication, laser light having a short wavelength can realize shaping with a higher resolution. Furthermore, in the specification of Blu-ray (registered trademark) disk, laser light having a central wavelength of 405 nm is used. As described above, development of a compound having an excellent two-photon absorption property for light in the same wavelength region as that of laser light having a short wavelength can greatly contribute to the development of the industry.

In order to apply a compound having a two-photon absorption property to an industrial use, a two-photon absorption material including the compound is required to sufficiently express the two-photon absorption property. In the present specification, a compound having a two-photon absorption property may be called a two-photon absorption compound. In order to sufficiently express the two-photon absorption property of a two-photon absorption material, it is desirable that the two-photon absorption property per one molecule of the two-photon absorption compound is high and the density of the two-photon absorption compound in the two-photon absorption material is high. Incidentally, a high two-photon absorption property per one molecule of a two-photon absorption compound means a large two-photon absorption cross-section of the two-photon absorption compound.

A two-photon absorption compound having a high two-photon absorption property per molecular size is suitable for improving the two-photon absorption property per unit volume of a two-photon absorption material. For example, a two-photon absorption compound having a small molecular size and a large two-photon absorption cross-section is suitable for improving the two-photon absorption cross-section per unit volume of a two-photon absorption material. Examples of the index of the two-photon absorption property per molecular size of a two-photon absorption compound include the two-photon absorption cross-section per unit weight of the two-photon absorption compound. In the present specification, the value of the two-photon absorption cross-section per unit weight of a two-photon absorption compound may be called a GM·mol/g value. The GM·mol/g value is a value calculated by dividing the two-photon absorption cross-section (GM) of a two-photon absorption compound by the molecular weight (g/mol) of the two-photon absorption compound.

Japanese Patent Nos. 5659189 and 5821661 disclose compounds having large two-photon absorption cross-sections for light with a wavelength of about 405 nm. Japanese Unexamined Patent Application Publication No. 2013-242939 discloses an optical information recording medium that can shorten the recording time when laser light having a wavelength of about 405 nm is used and a compound included in the optical information recording medium.

The present inventors have earnestly studied and as a result, have newly found that a compound represented by a formula (1) described below has a high nonlinear light absorption property for light having a wavelength in a short wavelength region, and the nonlinear light absorption material of the present disclosure has been accomplished. Specifically, the present inventors have found that the compound represented by the formula (1) has a large GM·mol/g value for light having a wavelength in a short wavelength region. In the present specification, a short wavelength region means a wavelength region including 405 nm, for example, a wavelength region of 390 nm or more and 420 nm or less.

### Outline of an Aspect According to the Present Disclosure

A nonlinear light absorption material according to a 1st aspect of the present disclosure includes a compound represented by the following formula (1) as a main component:

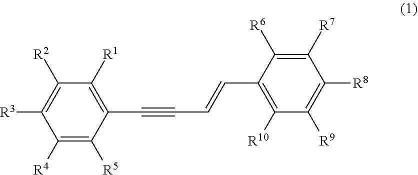

in the formula (1), R¹ to R¹⁰ are each independently a hydrogen atom, a halogen atom, a saturated hydrocarbon group, a halogenated alkyl group, an unsaturated hydrocarbon group, a hydroxyl group, a carboxyl group, an alkoxycarbonyl group, an aldehyde group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

According to the 1st aspect, the compound represented by the formula (1) tends to have a large GM·mol/g value for light having a wavelength in a short wavelength region. This compound is suitable for improving the two-photon absorption cross-section per unit volume of a nonlinear light absorption material. That is, a nonlinear light absorption material including a compound represented by the formula (1) is suitable for improving the nonlinear light absorption property for light having a wavelength in a short wavelength region. Furthermore, the compound represented by the formula (1) also tends to have a small molar absorption coefficient for light having a wavelength in a short wavelength region.

In a 2nd aspect of the present disclosure, for example, in the nonlinear light absorption material according to the 1st aspect, the compound may be represented by the following formula (2) or (3):

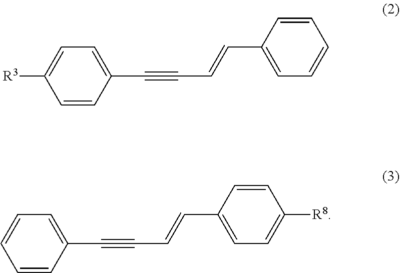

In a 3rd aspect of the present disclosure, for example, in the nonlinear light absorption material according to the 1st aspect, R¹ to R¹⁰ may be each a hydrogen atom.

In a 4th aspect of the present disclosure, for example, in the nonlinear light absorption material according to any one of the 1st to 3rd aspects, the compound may have a nonlinear light absorption effect.

In a 5th aspect of the present disclosure, for example, the nonlinear light absorption material according to any one of the 1st to 4th aspects may be used in a device using light having a wavelength of 390 nm or more and 420 nm or less.

The nonlinear light absorption material according to the 2nd to 5th aspects is suitable for improving the nonlinear light absorption property for light having a wavelength in a short wavelength region. This nonlinear light absorption material is suitable for application to a device using light having a wavelength of 390 nm or more and 420 nm or less.

A recording medium according to a 6th aspect of the present disclosure includes a recording layer including the nonlinear light absorption material according to any one of the 1st to 5th aspects.

According to a 6th aspect, the nonlinear light absorption material is suitable for improving the nonlinear light absorption property for light having a wavelength in a short wavelength region. A recording medium including such a nonlinear light absorption material can record information with a high recording density.

A recording method for recording information according to a 7th aspect of the present disclosure comprises:
preparing a light source emitting light having a wavelength of 390 nm or more and 420 nm or less; and
collecting the light from the light source and irradiating the recording layer of the recording medium including the nonlinear light absorption material according to the 6th aspect with the light.

According to the 7th aspect, the nonlinear light absorption material is suitable for improving the nonlinear light absorption property for light having a wavelength in a short wavelength region. According to the method for recording information using a recording medium including the nonlinear light absorption material, information can be recorded with a high recording density.

A reading method for reading information according to an 8th aspect of the present disclosure is a method for reading information recorded by, for example, the recording method according to the 7th aspect, the reading method includes:
irradiating the recording layer of the recording medium with light to measure an optical characteristic of the recording layer; and
reading the information from the recording layer.

In a 9th aspect of the present disclosure, for example, in the reading method for reading information according to the 8th aspect, the optical characteristic may be the intensity of light reflected by the recording layer.

According to the 8th or 9th aspect, information can be easily read.

Embodiment of the present disclosure will now be described with reference to the drawings. The present disclosure is not limited to the following embodiments.

### EMBODIMENTS

The nonlinear light absorption material of the present embodiment includes a compound A represented by the following formula (1):

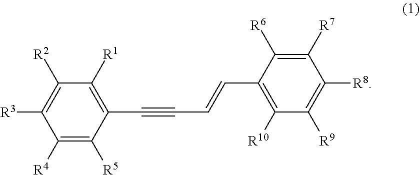

In the formula (1), R¹ to R¹⁰ each independently include at least one atom selected from the group consisting of H, C, N, O, F, P, S, Cl, I, and Br. R¹ to R¹⁰ may be each independently a hydrogen atom, a halogen atom, a saturated hydrocarbon group, a halogenated alkyl group, an unsaturated hydrocarbon group, a hydroxyl group, a carboxyl group, an alkoxycarbonyl group, an aldehyde group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

Examples of the halogen atom include F, Cl, Br, and I. In the present specification, the halogen atom may be called a halogen group.

The saturated hydrocarbon group is, for example, an aliphatic saturated hydrocarbon group. A specific example of the aliphatic saturated hydrocarbon group is an alkyl group. The number of carbon atoms of the alkyl group is not particularly limited and is, for example, 1 or more and 20 or less. The number of carbon atoms of the alkyl group may be 1 or more and 10 or less or 1 or more and 5 or less from the viewpoint of being capable of easily synthesizing the compound A. The solubility of the compound A in a solvent or resin composition can be adjusted by adjusting the number of carbon atoms of the alkyl group. The alkyl group may be linear, branched, or cyclic. At least one of the hydrogen atoms included in the alkyl group may be substituted by a group including at least one atom selected from the group consisting of N, O, P, and S. Examples of the alkyl group include a methyl group, an ethyl group, a propyl group, a butyl group, a 2-methylbutyl group, a pentyl group, a hexyl group, a 2,3-dimethylhexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a 2-methoxybutyl group, and a 6-methoxyhexyl group.

A halogenated alkyl group means a group in which at least one of the hydrogen atoms included in the alkyl group is substituted by a halogen atom. The halogenated alkyl group may be a group in which all the hydrogen atoms included in the alkyl group are substituted by halogen atoms. Examples of the alkyl group include those mentioned above. A specific example of the halogenated alkyl group is —CF₃.

The unsaturated hydrocarbon group has an unsaturated bond such as a carbon-carbon double bond and a carbon-carbon triple bond. The number of the unsaturated bonds included in the unsaturated hydrocarbon group is, for example, 1 or more and 5 or less. The number of carbon atoms of the unsaturated hydrocarbon group is not particularly limited and is, for example, 2 or more and 20 or less and may be 2 or more and 10 or less or 2 or more and 5 or less. The unsaturated hydrocarbon group may be linear, branched, or cyclic. At least one of hydrogen atoms included in the unsaturated hydrocarbon group may be substituted by a group including at least one atom selected from the group consisting of N, O, P, and S. Examples of the unsaturated hydrocarbon group include a vinyl group and an ethynyl group.

The hydroxyl group is represented by —OH. The carboxyl group is represented by —COOH. The alkoxycarbonyl group is represented by —COORₐ. The aldehyde group is represented by —COH. The acyl group is represented by —COR_{b}. The amide group is represented by —CONR_{c}R_{d}. The nitrile group is represented by —CN. The alkoxy group is represented by —ORₑ. The acyloxy group is represented by —OCOR_{f}. The thiol group is represented by —SH. The alkylthio group represented by —SR_{g}. The sulfonate group is represented by —SO₃H. The acylthio group is represented by —SCORₕ. The alkylsulfonyl group is represented by —SO₂Rᵢ. The sulfonamide group is represented by —SO₂NRⱼRₖ. The primary amino group is represented by —NH₂. The secondary amino group is represented by —NHRₗ. The tertiary amino group is represented by —NRₘRₙ. The nitro group is represented by —NO₂. Rₐ to Rₙ are each independently an alkyl group. Examples of the alkyl group include those mentioned above. However, the amide groups R_{c} and R_{d} and sulfonamide groups Rⱼ and Rₖ may be each independently a hydrogen atom.

Specific examples of the alkoxycarbonyl group are —COOCH₃, —COO(CH₂)₃CH₃, and —COO(CH₂)₇CH₃. A specific example of the acyl group is —COCH₃. A specific example of the amide group is —CONH₂. Specific examples of the alkoxy group are a methoxy group, an ethoxy group, a 2-methoxyethoxy group, a butoxy group, a 2-methylbutoxy group, a 2-methoxybutoxy group, a 4-ethylthiobutoxy group, a pentyloxy group, a hexyloxy group, a heptyloxy group, an octyloxy group, a nonyloxy group, a decyloxy group, an undecyloxy group, a dodecyloxy group, a tridecyloxy group, a tetradecyloxy group, a pentadecyloxy group, a hexadecyloxy group, a heptadecyloxy group, an octadecyloxy group, a nonadecyoxy group, and an eicosyloxy group. A specific example of the acyloxy group is —OCOCH₃. A specific example of the acylthio group is —SCOCH₃. A specific example of the alkylsulfonyl group is —SO₂CH₃. A specific example of the sulfonamide group is —SO₂NH₂. A specific example of the tertiary amino group is —N(CH₃)₂.

In the formula (1), at least one selected from the group consisting of R³ and R⁸ may be an electron donating group or an electron withdrawing group. Regarding R³ or R⁸, the higher the electron donating property or electron withdrawing property, the larger the electron deviation in the compound A. When the electron deviation in the compound A is large, the electrons tend to largely move in the compound A by exciting the compound A. Such a compound A tends to have a more excellent two-photon absorption property. In other words, when at least one selected from the group consisting of R³ and R⁸ is an electron donating group or an electron withdrawing group, the compound A tends to have a large two-photon absorption cross-section. However, R³ and R⁸ may be each a hydrogen atom.

An electron withdrawing group means, for example, a substituent having a positive substituent constant σₚ value in the Hammett equation. Examples of the electron withdrawing group include halogen atoms, a carboxyl group, a nitro group, a thiol group, a sulfonate group, an acyloxy group, an alkylthio group, an alkylsulfonyl group, a sulfonamide group, an acyl group, an acylthio group, an alkoxycarbonyl group, and a halogenated alkyl group. The electron withdrawing group may be a carboxyl group or an alkoxycarbonyl group and may be —COO(CH₂)₃CH₃ or —COO(CH₂)₇CH₃.

The electron donating group means, for example, a substituent having a negative σₚ value. Examples of the electron donating group include an alkyl group, an alkoxy group, hydroxyl group, and an amino group.

In the formula (1), R¹, R⁵, R⁶, and R¹⁰ may each have a small volume. On this occasion, in R¹, R⁵, R⁶, and R¹⁰, steric hindrance is unlikely to be caused. Accordingly, in the compound A, the planarity of π electron conjugated system tends to be improved. When the π electron conjugated system of the compound A has a high planarity, the compound A tends to have a large two-photon absorption cross-section. R¹, R⁵, R⁶, and R¹⁰ may be each a hydrogen atom.

Furthermore, R¹, R², and R⁴ to R¹⁰ may be each a hydrogen atom, or R¹ to R⁷, R⁹, and R¹⁰ may be each a hydrogen atom. That is, the compound A may be a compound B represented by the following formula (2) or a compound C represented by the following formula (3):

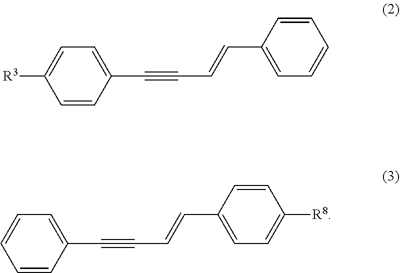

R³ in the formula (2) and R⁸ in the formula (3) are the same as those described in the formula (1). Table 1 below shows specific examples of R³ in the formula (2) and R⁸ in the formula (3). In the formula (2), R³ may be —H. That is, in the formula (1), R¹ to R¹⁰ may be each a hydrogen atom.

TABLE 1

| | R³ and R⁸ |
|---|---|
| 1 | —H |
| 2 | —F |
| 3 | —CH₃ |
| 4 | —C₂H₅ |
| 5 | —CF₃ |
| 6 | —OH |
| 7 | —COOH |
| 8 | —COOCH₃ |
| 9 | —COCH₃ |
| 10 | —CONH₂ |
| 11 | —CN |
| 12 | —OCH₃ |
| 13 | —OCOCH₃ |
| 14 | —SH |
| 15 | —SO₃H |
| 16 | —SCOCH₃ |
| 17 | —SO₂CH₃ |
| 18 | —SO₂NH₂ |
| 19 | —NH₂ |
| 20 | —N(CH₃)₂ |
| 21 | —NO₂ |

The synthesis method of the compound B represented by the formula (2) is not particularly limited. The compound B can be synthesized by, for example, the following method. First, a compound D represented by the following formula (4) is prepared. In the formula (4), R³ is the same as that described for the formula (1) above.

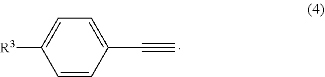

Then, a coupling reaction between the compound D and β3-bromostyrene is performed. Consequently, the compound B can be synthesized. The conditions for the coupling reaction can be appropriately adjusted according to, for example, the structure of the compound D. Incidentally, the compound C of the formula (3) can be synthesized by performing the same coupling reaction as that in the synthesis method of the compound B.

The compound A represented by the formula (1) has an excellent two-photon absorption property for light having a wavelength in a short wavelength region and tends to be low in one-photon absorption. As an example, when the compound A is irradiated with light having a wavelength of 405 nm, in the compound A, one-photon absorption may be little caused while two-photon absorption is caused.

The two-photon absorption cross-section of the compound A for light having a wavelength of 405 nm may be higher than 1 GM, 10 GM or more, 100 GM or more, or higher than 200 GM. The upper limit of the two-photon absorption cross-section of the compound A is not particularly limited and may be, for example, 5000 GM and may be 1000 GM. The two-photon absorption cross-section can be measured by, for example, the Z-scan method described in J. Opt. Soc. Am. B, 2003, Vol. 20, p. 529. The Z-scan method is broadly used as a method for measuring a nonlinear optical constant. In the Z-scan method, a measurement sample is moved along the irradiation direction of a laser beam near the focal point where the beam is focused. On this occasion, the change in the amount of light permeated through the measurement sample is recorded. In the Z-scan method, the power density of incident light changes depending on the position of the measurement sample. Accordingly, when the measurement sample performs nonlinear light absorption, if the measurement sample is located near the focal point of the laser beam, the amount of the transmitted light is attenuated. The two-photon absorption cross-section can be calculated by fitting the change of the amount of transmitted light to a theoretical curve predicted by the intensity of incident light, the thickness of the measurement sample, the concentration of the compound A in the measurement sample, and so on.

The two-photon absorption cross-section may be a calculated value by computational chemistry. Several methods for estimating the two-photon absorption cross-section by computational chemistry have been proposed. For example, the calculated value of a two-photon absorption cross-section can be calculated based on a second-order nonlinear response theory described in J. Chem. Theory Comput., 2018, Vol. 14, p. 807.

In the present embodiment, the value (GM·mol/g value) of the two-photon absorption cross-section (GM) per unit weight of the compound A for light having a wavelength of 405 nm tends to be large. The compound A may have a GM·mol/g value of 0.9 or more, 1.0 or more, 1.5 or more, or 2.0 or more. The upper limit of the GM·mol/g value of the compound A is not particularly limited and is, for example, 50.

The molar absorption coefficient of the compound A for light having a wavelength of 405 nm may be 100 mol⁻¹·L·cm⁻¹ or less, 10 mol⁻¹·L·cm⁻¹ or less, 5 mol⁻¹·L·cm⁻¹ or less, 1 mol⁻¹·L·cm⁻¹ or less, or 0.1 mol⁻¹·L·cm⁻¹ or less. The lower limit of the molar absorption coefficient of the compound A is not particularly limited and is, for example, 0.00001 mol⁻¹·L·cm⁻¹. The molar absorption coefficient can be measured by, for example, a method in accordance with the provision of Japanese Industrial Standards (JIS) K0115:2004. The measurement of molar absorption coefficient uses a light source that irradiates light with a photon density to hardly cause two-photon absorption by the compound A. Furthermore, in the measurement of molar absorption coefficient, the concentration of the compound A is adjusted to 1 mmol/L or more and 50 mmol/L or less. This concentration is very high compared to the concentration in a measurement test of the molar absorption coefficient of a light absorption peak. The molar absorption coefficient can be used as an index of one-photon absorption.

The molar absorption coefficient may be a calculated value by a quantum chemical calculation program. As the quantum chemical calculation program, for example, Gaussian 16 (manufactured by Gaussian, Inc.) can be used.

When the compound A performs two-photon absorption, the compound A absorbs energy about twice that of light applied to the compound A. The wavelength of light having energy about twice that of the light having a wavelength of 405 nm is, for example, 200 nm. When the compound A is irradiated with light having a wavelength of about 200 nm, one-photon absorption may occur in the compound A. Furthermore, in the compound A, one-photon absorption may occur for light having a wavelength near the wavelength region where two-photon absorption is caused.

The nonlinear light absorption material of the present embodiment may contain the compound A represented by the formula (1) as a main component. The term “main component” means the most abundant component by weight included in the nonlinear light absorption material. The nonlinear light absorption material, for example, substantially consists of the compound A. The phrase “substantially consist of” means to exclude other components that change the essential characteristics of the material referred to. However, the nonlinear light absorption material may include impurities in addition to the compound A. The nonlinear light absorption material including the compound A of the present embodiment functions as, for example, a two-photon absorption material.

The nonlinear light absorption material of the present embodiment is used in, for example, a device using light having a wavelength in a short wavelength region. As an example, the nonlinear light absorption material of the present embodiment is used in a device using light having a wavelength of 390 nm or more and 420 nm or less. Examples of the device include a recording medium, a shaping device, and a fluorescence microscope. Examples of the recording medium include a three-dimensional optical memory. A specific example of the three-dimensional optical memory is a three-dimensional optical disk. Examples of the shaping device include a three-dimensional (3d) laser microfabrication device such as a 3D printer. Examples of the fluorescence microscope include a two-photon fluorescence microscope. The light used in these devices has, for example, a high photon density near the focal point. The power density near the focal point of the light that is used in the device is, for example, 0.1 W/cm² or more and 1.0×10²⁰ W/cm² or less. The power density near the focal point of this light may be 1.0 W/cm² or more, 1.0×10² W/cm² or more, or 1.0×10⁵ W/cm² or more. As the light source of the device, for example, it is possible to use a femtosecond laser such as a titanium sapphire laser or a pulsed laser having a picosecond to nanosecond pulse width such as a semiconductor laser.

The recording medium includes, for example, a thin film called a recording layer. In the recording medium, information is recorded in the recording layer. As an example, a thin film as the recording layer includes the nonlinear light absorption material of the present embodiment. That is, from another aspect, the present disclosure provides a recording medium including a nonlinear light absorption material including the compound A above.

The recording layer may further include a polymer compound that functions as a binder, in addition to the nonlinear light absorption material. The recording medium may include a dielectric layer in addition to the recording layer. The recording medium includes, for example, a plurality of recording layers and a plurality of dielectric layers. In the recording medium, a plurality of recording layers and a plurality of dielectric layers may be alternately stacked.

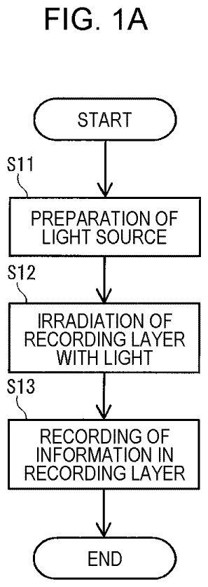

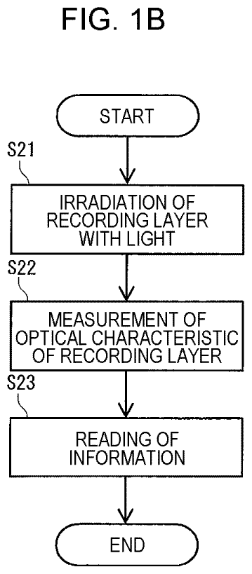

Then, a method for recording information using the recording medium above will be described. FIG. 1A is a flow chart of a method for recording information using the recording medium. First, in step S11, a light source that emits light having a wavelength of 390 nm or more and 420 nm or less is prepared. As the light source, for example, it is possible to use a femtosecond laser such as a titanium sapphire laser or a pulsed laser having a picosecond to nanosecond pulse width such as a semiconductor laser. Then, in step S12, the light from the light source is condensed with a lens or the like and is applied to the recording layer of the recording medium. Specifically, the light from the light source is condensed with a lens or the like and is applied to the recording area of the recording medium. The power density near the focal point of this light is, for example, 0.1 W/cm² or more and 1.0×10²⁰ W/cm² or less. This power density near the focal point of the light may be 1.0 W/cm² or more, 1.0×10² W/cm² or more, or 1.0×10⁵ W/cm² or more. In the present specification, the recording area means a spot that is present in a recording layer and can record information by being irradiated with light.

In the recording area irradiated with light, a physical change or a chemical change occurs. For example, heat is generated when the compound A absorbed light turns from the transition state to the ground state. This heat changes the nature of the binder present in the recording area. Consequently, the optical characteristics of the recording area change. The changes are, for example, the intensity of light reflected at the recording area, the reflectance of light at the recording area, the absorptance of light at the recording area, and the refractive index of light in the recording area. In the recording area irradiated with light, the intensity of fluorescent light emitted from the recording area or the wavelength of fluorescent light may also change. Consequently, information can be recorded in the recording layer, specifically, in the recording area (step S13).

Then, a method for reading information using the recording medium will be described. FIG. 1B is a flow chart of a method for reading information using the recording medium. First, in step S21, the recording layer of the recording medium is irradiated with light. Specifically, the recording area of the recording medium is irradiated with light. The light used in step S21 may be the same as or different from the light used for recording information on the recording medium. Then, in step S22, an optical characteristic of the recording layer is measured. Specifically, an optical characteristic of the recording area is measured. In step S22, for example, as the optical characteristic of the recording area, the intensity of light reflected at the recording area is measured. In step S22, as the optical characteristic of the recording area, for example, the reflectance of light at the recording area, the absorptance of light at the recording area, the refractive index of light in the recording area, the intensity of fluorescent light emitted from the recording area, or the wavelength of fluorescent light may be measured. Then, in step S23, information is read from the recording layer, specifically, from the recording area.

In the method for reading information, the recording area where information is recorded can be located by the following method. First, a specific area of the recording medium is irradiated with light. This light may be the same as or different from the light used for recording information on the recording medium. Then, an optical characteristic of the area irradiated with the light is measured. Examples of the optical characteristic include the intensity of light reflected at the area, the reflectance of light at the area, the absorptance of light at the area, the refractive index of light in the area, the intensity of fluorescent light emitted from the area, and the wavelength of fluorescent light emitted from the area. Whether an area irradiated with light is a recording area or not is judged based on the measured optical characteristic. For example, when the intensity of light reflected at an area is a specific value or less, the area is judged to be a recording area. In contrast, when the intensity of light reflected at an area exceeds a specific value, the area is judged not to be a recording area. Incidentally, the method for judging whether an area irradiated with light is a recording area or not is not limited to the above method. For example, when the intensity of light reflected at an area exceeds a specific value, the area may be judged to be a recording area. Alternatively, when the intensity of light reflected at an area is a specific value or less, the area may be judged not to be a recording area. When an area is judged not to be a recording area, the same procedure is performed for another area of the recording medium. Consequently, a recording area can be located.

The recording method and reading method of information using the recording medium can be performed with, for example, a known recording apparatus. The recording apparatus includes, for example, a light source for irradiating the recording area of a recording medium with light, a measuring device of measuring an optical characteristic of the recording area, and a controller for controlling the light source and the measuring device.

The shaping device performs shaping by, for example, irradiating a photocurable resin composition with light to cure the resin composition. As an example, a photocurable resin composition for three-dimensional (3d) laser microfabrication includes a nonlinear light absorption material of the present embodiment. The photocurable resin composition includes, for example, a polymerizable compound and a polymerization initiator, in addition to the nonlinear light absorption material. The photocurable resin composition may further include an additive such as a binder resin. The photocurable resin composition may include an epoxy resin.

A fluorescence microscope can, for example, irradiate a biological specimen including a fluorescent dye material with light and observe fluorescence emitted from the dye material. As an example, the fluorescent dye material to be added to a biological specimen includes the nonlinear light absorption material of the present embodiment.

### EXAMPLES

The present disclosure will now be described in more detail by examples. Incidentally, the following examples are merely examples, and the present disclosure is not limited to the following examples. In the present disclosure, a compound used in an example is written as “compound (X)—Y”. “X” means the structural formula of the compound. “Y” means the type of substituent R³ or R⁸ in the formula (X). The value of “Y” corresponds to Table 1. For example, compound (2)-1 means a compound represented by formula (2) and having substituent 1 (—H) shown in Table 1 as R³.

### Synthesis of Compound (2)-1

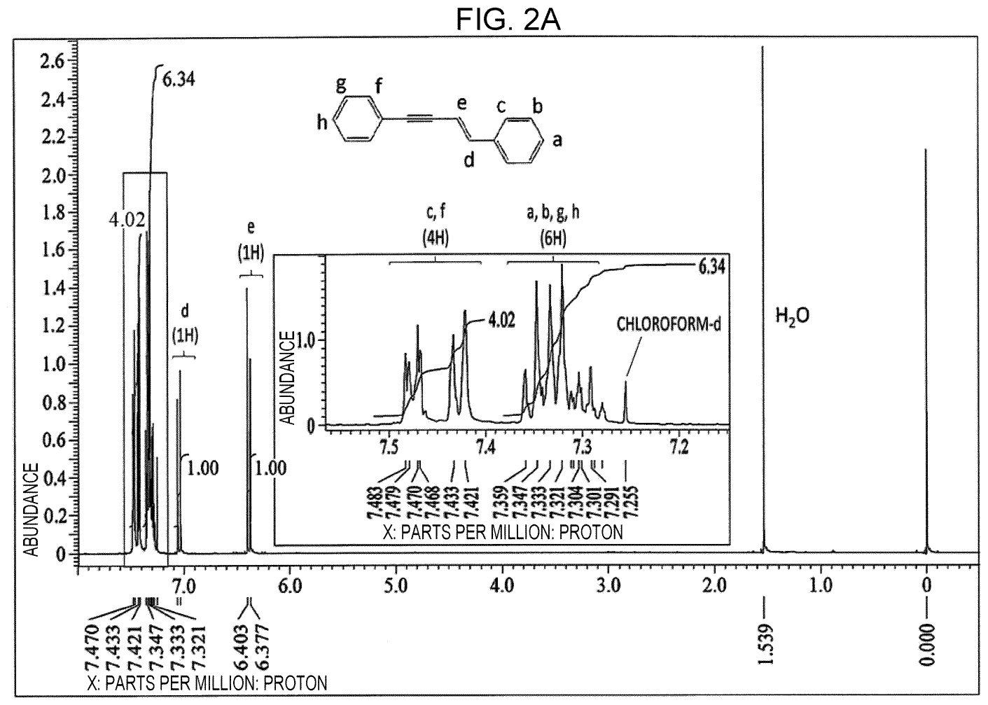

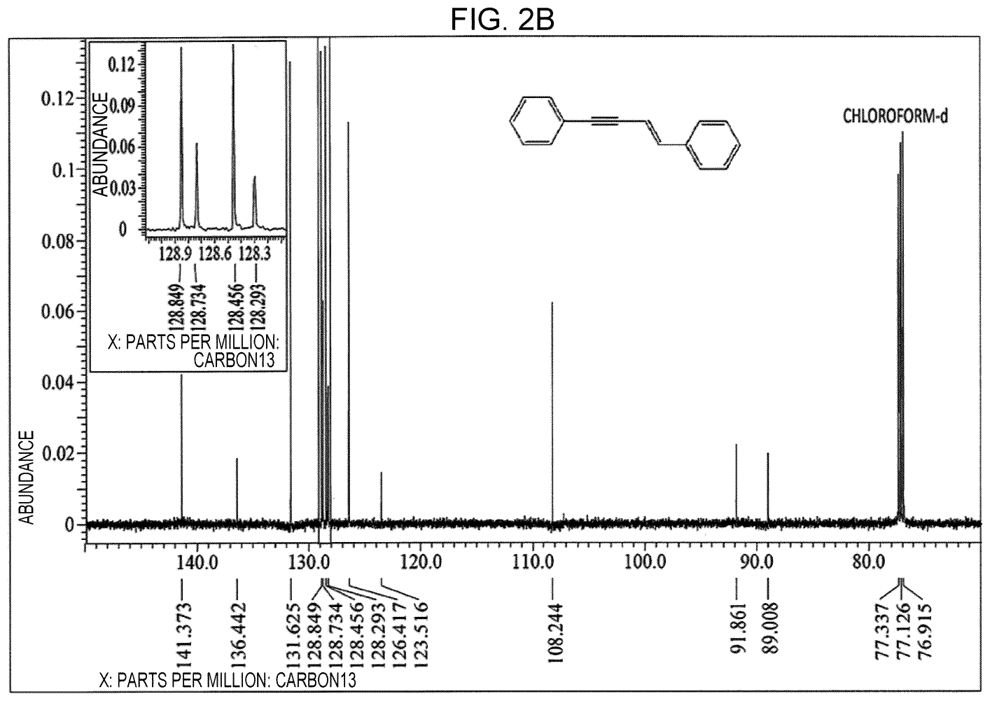

First, triphenylphosphine (manufactured by Tokyo Chemical Industry Co., Ltd.), potassium carbonate (manufactured by FUJIFILM Wako Pure Chemical Corporation), tetrabutylammonium acetate (manufactured by Tokyo Chemical Industry Co., Ltd.), and copper(I) iodide (manufactured by FUJIFILM Wako Pure Chemical Corporation) were charged in a reaction vessel, and the inside of the vessel was replaced with argon. Then, ion exchanged water, ethynylbenzene (manufactured by Tokyo Chemical Industry Co., Ltd.), and β-bromostyrene (manufactured by Sigma-Aldrich Co. LLC) were injected into this reaction vessel, followed by stirring at 110° C. for 19 hours. The obtained reaction solution was subjected to extraction treatment using ethyl acetate (manufactured by FUJIFILM Wako Pure Chemical Corporation). The obtained extraction liquid was washed with saturated saline and was then dehydrated by addition of magnesium sulfate. Furthermore, the extraction liquid was concentrated using a rotary evaporator. The obtained concentrate was purified by silica gel column chromatography to obtain compound (2)-1. The compound (2)-1 was identified by ¹H-NMR and ¹³C-NMR. FIG. 2A is a graph showing the ¹H-NMR spectrum of the compound (2)-1. FIG. 2B is a graph showing the ¹³C-NMR spectrum of the compound (2)-1. Incidentally, in FIG. 2A, the integrated value (4.02) of peaks within a range of 7.4 ppm or more and 7.5 ppm or less overlaps with other peaks. However, this integrated value and peaks can be clearly read from an enlarged view of the central part of FIG. 2A. The ¹H-NMR spectrum and ¹³C-NMR spectrum of the compound (2)-1 are as follows:

¹H-NMR (600 MHz, CHLOROFORM-D) δ 7.42-7.48 (m, 4H), 7.28-7.36 (m, 6H), 7.05 (d, J=16.5 Hz, 1H), 6.39 (d, J=15.8 Hz, 1H);

¹³C-NMR (151 MHz, CHLOROFORM-D) δ 141.37, 136.44, 131.63, 128.85, 128.73, 128.46, 128.29, 126.42, 123.52, 108.24, 91.86, 89.01.

### Comparative Examples 1 to 3

Furthermore, compounds of Comparative Examples 1 to 3 shown in Table 3 were prepared. The compounds of Comparative Examples 1 to 3 are represented by the following formulae (5) to (7), respectively.

Here, the compound D29 used as the compound of Comparative Example 1 represented by the following formula (5) was synthesized according to the method described in paragraphs [0222] to [0230] of Japanese Patent No. 5659189. The compound if used as the compound of Comparative Example 2 represented by the following formula (6) was synthesized according to the method described in paragraph [0083] of Japanese Patent No. 5821661. The DPB used as the compound of Comparative Example 3 was that manufactured by Tokyo Chemical Industry Co., Ltd.

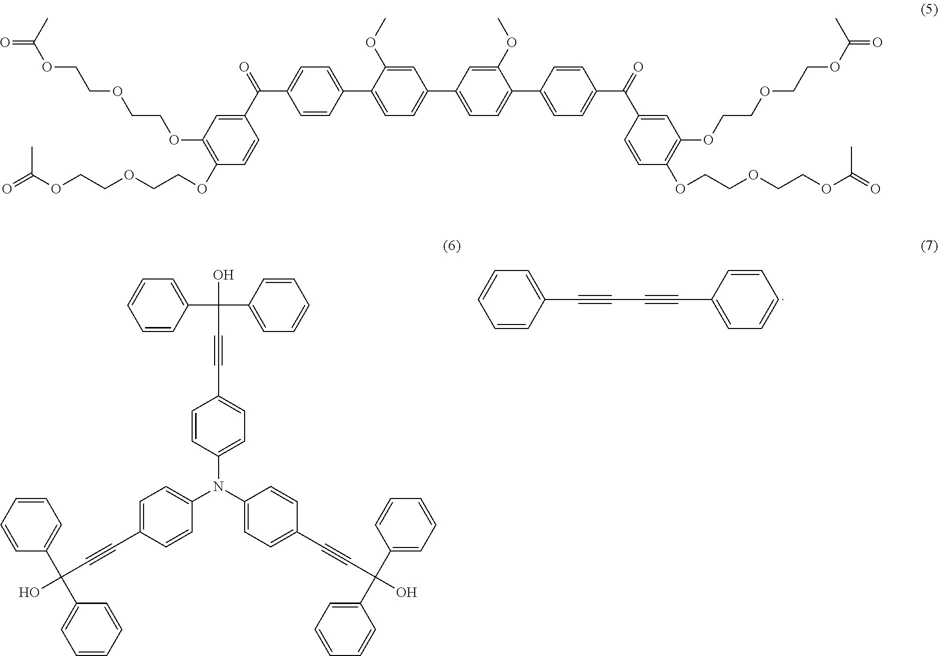

### Measurement Two-Photon Absorption Cross-Section

The synthesized compounds and the compounds of Comparative Examples were measured for the two-photon absorption cross-section for light having a wavelength of 405 nm. The measurement of the two-photon absorption cross-section was performed using the Z-scan method described in J. Opt. Soc. Am. B, 2003, Vol. 20, p. 529. As the light source for measuring the two-photon absorption cross-section, a titanium sapphire pulsed laser was used. Specifically, each sample was irradiated with the second harmonic of the titanium sapphire pulsed laser. The pulse width of the laser was 80 fs. The repetition frequency of the laser was 1 kHz. The average power of the laser was changed within a range of 0.01 mW or more and 0.08 mW or less. The light from the laser was light having a wavelength of 405 nm. Specifically, the light from the laser had a central wavelength of 403 nm or more and 405 nm or less. The light from the laser had a full width at half maximum of 4 nm.

### Prediction of Two-Photon Absorption Cross-Section

The synthesized compounds and the compounds of Comparative Examples were predicted for the two-photon absorption cross-section for light having a wavelength of 405 nm. Specifically, the two-photon absorption cross-section was calculated by a density functional theory (DFT) calculation based on the second-order nonlinear response theory described in J. Chem. Theory Comput., 2018, Vol. 14, p. 807. In the DFT calculation, as software, Turbomole version 7.3.1 (manufactured by COSMOlogic GmbH & Co. KG) was used. As the basis function, def2-TZVP was used. As the generic function, B3LYP was used.

The calculated and measured values of the two-photon absorption cross-section of the synthesized compounds and the compounds of Comparative Examples were subjected to linear regression. Then, using a regression equation obtained from this linear regression, the calculated value of the two-photon absorption cross-section was calculated for another compound different from the synthesized compounds in the type of the substituent, the position of the substituent, and so on.

### Measurement of Molar Absorption Coefficient

The synthesized compounds and the compounds of Comparative Examples were measured for the molar absorption coefficient by a method in accordance with the provision of JIS K0115:2004. Specifically, first, as a measurement sample, a solution in which a compound was dissolved in a solvent was prepared. The concentration of the compound in the solution was appropriately adjusted within a range of 1 mmol/L or more and 50 mmol/L or less according to the absorbance at a wavelength of 405 nm of the compound as the target for measurement. Then, the absorption spectrum of the measurement sample was measured. The absorbance at a wavelength of 405 nm was read from the obtained spectrum. The molar absorption coefficient was calculated based on the concentration of the compound in the measurement sample and the optical path length of the cell used for the measurement.

### Prediction of Molar Absorption Coefficient

The synthesized compounds and the compounds of Comparative Examples were predicted for the molar absorption coefficient. In the prediction of molar absorption coefficient, DFT calculation was adopted. Specifically, first, the excited state was calculated for the compound using a quantum chemical calculation program, Gaussian 16 (manufactured by Gaussian, Inc.). In the excited state calculation, as the basis function, 6-31++G (d,p) was used. As the generic function, B3LYP was used. The energy for exciting a compound and oscillator strength f were calculated by the excited state calculation. The oscillator strength correlates with the molar absorption coefficient. Then, the half-value width was defined by assuming the absorption spectrum to be Gaussian distribution. Specifically, an absorption spectrum was drawn with the half-width value defined as 0.4 eV based on the absorption wavelength and oscillator strength. The absorbance at a wavelength of 405 nm was read from the obtained absorption spectrum. This absorbance was recognized as the calculated value of the molar absorption coefficient.

The calculated and measured values of the molar absorption coefficients of the synthesized compounds and the compounds of Comparative Examples were subjected to linear regression. In the linear regression, the coefficient of determination, R² value, exceeded 0.9. This revealed that there was a high correlation between the calculated value and the measured value of molar absorption coefficient. Then, using a regression equation obtained from this linear regression, the calculated value of the molar absorption coefficient was calculated for another compound different from the synthesized compounds in the type of the substituent, the position of the substituent, and so on was calculated.

The measured and calculated values of two-photon absorption cross-section (GM) and the measured and calculated values of molar absorption coefficient (mol⁻¹·L·cm⁻¹) obtained by the methods above and GM·mol/g values are shown in Tables 2 and 3. In Tables 2 and 3, the GM·mol/g values were calculated based on the measured values of two-photon absorption cross-section. In a compound of which the measured value of two-photon absorption cross-section was not obtained, the GM·mol/g value was calculated based on the calculated value of two-photon absorption cross-section. In Tables 2 and 3, “No Data” means that no data was obtained.

TABLE 2

| | | | Two-photon | | | Molar absorption | |
|---|---|---|---|---|---|---|---|
| | | | absorption cross- | | | coefficient | |
| | | Molecular | section (GM) | | GM · | (mol⁻¹ · L · cm⁻¹) | |
| | | weight | Measured | Calculated | mol/g | Measured | Calculated |
| | Compound | (g/mol) | value | value | value | value | value |
| Example 1 | (2)-1 | 204.27 | 320 ± 30 | 275 | 1.57 | 0.54 | 0.49 |
| Example 2 | (2)-2 | 222.26 | No Data | 285 | 1.28 | No Data | 0.51 |
| Example 3 | (2)-3 | 218.30 | No Data | 460 | 2.11 | No Data | 0.86 |
| Example 4 | (2)-4 | 232.33 | No Data | 335 | 1.44 | No Data | 0.96 |
| Example 5 | (2)-5 | 272.27 | No Data | 355 | 1.30 | No Data | 0.95 |
| Example 6 | (2)-6 | 220.27 | No Data | 230 | 1.04 | No Data | 1.09 |
| Example 7 | (2)-7 | 248.28 | No Data | 350 | 1.41 | No Data | 3.44 |
| Example 8 | (2)-8 | 262.31 | No Data | 345 | 1.32 | No Data | 3.10 |
| Example 9 | (2)-9 | 246.31 | No Data | 2470 | 10.0 | No Data | 4.41 |
| Example 10 | (2)-10 | 247.30 | No Data | 2400 | 9.7 | No Data | 2.23 |
| Example 11 | (2)-11 | 229.28 | No Data | 1225 | 5.34 | No Data | 2.84 |
| Example 12 | (2)-12 | 234.30 | No Data | 275 | 1.17 | No Data | 1.52 |
| Example 13 | (2)-13 | 262.31 | No Data | 260 | 0.99 | No Data | 0.84 |
| Example 14 | (2)-14 | 236.34 | No Data | 310 | 1.31 | No Data | 2.50 |
| Example 15 | (2)-15 | 284.34 | No Data | 290 | 1.02 | No Data | 2.02 |
| Example 16 | (2)-16 | 278.38 | No Data | 330 | 1.19 | No Data | 1.88 |
| Example 17 | (2)-17 | 282.36 | No Data | 275 | 0.97 | No Data | 1.60 |
| Example 18 | (2)-18 | 283.35 | No Data | 310 | 1.09 | No Data | 1.61 |
| Example 19 | (2)-19 | 219.29 | No Data | 295 | 1.34 | No Data | 3.01 |
| Example 20 | (2)-20 | 247.34 | No Data | 595 | 2.41 | No Data | 6.00 |
| Example 21 | (2)-21 | 249.27 | No Data | 370 | 1.48 | No Data | 4.00 |

TABLE 3

| | | | Two-photon | | | Molar absorption | |
|---|---|---|---|---|---|---|---|
| | | | absorption cross- | | | coefficient | |
| | | Molecular | section (GM) | | GM · | (mol⁻¹ · L · cm⁻¹) | |
| | | weight | Measured | Calculated | mol/g | Measured | Calculated |
| | Compound | (g/mol) | value | value | value | value | value |
| Example 22 | (3)-2 | 222.26 | No Data | 370 | 1.66 | No Data | 0.55 |
| Example 23 | (3)-3 | 218.30 | No Data | 260 | 1.19 | No Data | 0.78 |
| Example 24 | (3)-4 | 232.33 | No Data | 330 | 1.42 | No Data | 0.82 |
| Example 25 | (3)-5 | 272.27 | No Data | 265 | 0.97 | No Data | 1.18 |
| Example 26 | (3)-6 | 220.27 | No Data | 360 | 1.63 | No Data | 0.96 |
| Example 27 | (3)-7 | 248.28 | No Data | 535 | 2.19 | No Data | 3.94 |
| Example 28 | (3)-8 | 262.31 | No Data | 405 | 1.54 | No Data | 3.55 |
| Example 29 | (3)-9 | 246.31 | No Data | 345 | 1.40 | No Data | 4.90 |
| Example 30 | (3)-10 | 247.30 | No Data | 285 | 1.15 | No Data | 2.58 |
| Example 31 | (3)-11 | 229.28 | No Data | 490 | 2.14 | No Data | 3.49 |
| Example 32 | (3)-12 | 234.30 | No Data | 540 | 2.30 | No Data | 1.26 |
| Example 33 | (3)-13 | 262.31 | No Data | 425 | 1.62 | No Data | 1.01 |
| Example 34 | (3)-14 | 236.34 | No Data | 235 | 0.99 | No Data | 2.62 |
| Example 35 | (3)-15 | 284.34 | No Data | 260 | 0.91 | No Data | 2.59 |
| Example 36 | (3)-16 | 278.38 | No Data | 350 | 1.26 | No Data | 2.34 |
| Example 37 | (3)-17 | 282.36 | No Data | 360 | 1.28 | No Data | 2.07 |
| Example 38 | (3)-18 | 283.35 | No Data | 520 | 1.84 | No Data | 2.03 |
| Example 39 | (3)-19 | 219.29 | No Data | 350 | 1.60 | No Data | 1.43 |
| Example 40 | (3)-20 | 247.34 | No Data | 375 | 1.52 | No Data | 5.71 |
| Example 41 | (3)-21 | 249.27 | No Data | 350 | 1.40 | No Data | 3.74 |
| Comparative | D-29 (5) | 1159.24 | 570 ± 150 | 415 | 0.49 | 35 | 26.4 |
| Example 1 | | | | | | | |
| Comparative | If (6) | 864.06 | 380 ± 50 | 410 | 0.44 | 70 | 53 |
| Example 2 | | | | | | | |
| Comparative | DPB (7) | 202.26 | 160 ± 60 | 200 | 0.79 | 0.27 | 0.33 |
| Example 3 | | | | | | | |

As obvious from Tables 2 and 3, in all the compounds of Examples 1 to 41 corresponding to the compound A represented by the formula (1), the two-photon absorption cross-section value (GM·mol/g value) per unit weight for light having a wavelength of 405 nm was larger than those of the compounds of Comparative Examples and exceeded 0.9. This result demonstrates that the compound A is suitable for improving the two-photon absorption cross-section per unit volume of a nonlinear light absorption material. That is, it is demonstrated that a nonlinear light absorption material containing the compound A is suitable for improving the nonlinear light absorption property for light having a wavelength in a short wavelength region. Furthermore, in the compounds of Examples 1 to 41, the value of molar absorption coefficient for light having a wavelength of 405 nm was lower than 10, i.e., a relatively low value. Thus, it is demonstrated that the compound A expresses an excellent nonlinear light absorption property while having a small molecular size.

In the compound A represented by the formula (1), two benzene rings are linked by a linker in which carbon-carbon double bonds and carbon-carbon triple bonds are sequentially arranged. It is inferred that a transition dipole moment between a plurality of excited states was increased in the compound A due to its structure to increase the efficiency of two-photon absorption and that, consequently, a large GM·mol/g value and a low molar absorption coefficient were compatible with each other in the compounds of Examples.

The compounds of Comparative Examples 1 to 3 are compounds different from the compound A. In the compounds of Comparative Examples 1 to 3, since the GM·mol/g values for light having a wavelength of 405 nm are small, a large GM·mol/g value and a small molar absorption coefficient were incompatible with each other. Since the compounds of Comparative Examples 1 and 2 have large π-electron conjugated systems, the transition dipole moments are large. Accordingly, in Comparative Examples 1 and 2, the two-photon absorption cross-section values were relatively large. However, in the compounds of Comparative Examples 1 and 2, the GM·mol/g values were small due to the large molecular weights. Furthermore, in a compound having an extended π-electron conjugated system, the peak derived from one-photon absorption tends to shift to the longer wavelength region. It is inferred that in the compounds of Comparative Examples 1 and 2, the wavelength region where one-photon absorption occurs partially overlaps with 405 nm to significantly increase the molar absorption coefficient F.

The nonlinear light absorption material of the present disclosure can be used in applications, such as a recording layer of a three-dimensional optical memory and a photocurable resin composition for three-dimensional (3d) laser microfabrication. The nonlinear light absorption material of the present disclosure has a light absorption property exhibiting high nonlinearity for light having a wavelength in a short wavelength region. Accordingly, the nonlinear light absorption material of the present disclosure can achieve a significantly high spatial resolution in application such as a three-dimensional optical memory and a shaping device.

What is claimed is:

1. A nonlinear light absorption material comprising:
a compound represented by a formula (1) as a main component: 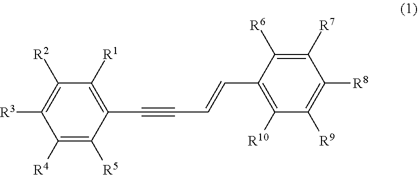
in the formula (1), R¹ to R¹⁰ are each independently a hydrogen atom, a halogen atom, a saturated hydrocarbon group, a halogenated alkyl group, an unsaturated hydrocarbon group, a hydroxyl group, a carboxyl group, an alkoxycarbonyl group, an aldehyde group, an acyl group, an amide group, a nitrile group, an alkoxy group, an acyloxy group, a thiol group, an alkylthio group, a sulfonate group, an acylthio group, an alkylsulfonyl group, a sulfonamide group, a primary amino group, a secondary amino group, a tertiary amino group, or a nitro group.

2. The nonlinear light absorption material according to claim 1, wherein
the compound is represented by a formula (2) or (3):

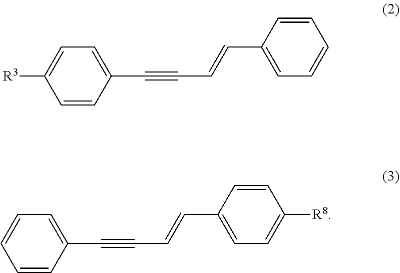

3. The nonlinear light absorption material according to claim 1, wherein
R¹ to R¹⁰ are each a hydrogen atom.

4. The nonlinear light absorption material according to claim 1, wherein
the compound has a nonlinear light absorption effect.

5. The nonlinear light absorption material according to claim 1, wherein
the nonlinear light absorption material is used in a device using light having a wavelength of 390 nm or more and 420 nm or less.

6. A recording medium comprising:
a recording layer including the nonlinear light absorption material according to claim 1.

7. A recording method for recording information, the recording method comprising:
preparing a light source emitting light having a wavelength of 390 nm or more and 420 nm or less; and
collecting the light from the light source and irradiating the recording layer of the recording medium including the nonlinear light absorption material according to claim 6 with the light.

8. A reading method for reading information recorded by the recording method according to claim 7, the reading method comprising:
irradiating the recording layer of the recording medium with light to measure an optical characteristic of the recording layer; and
reading the information from the recording layer.

9. The reading method according to claim 8, wherein
the optical characteristic is intensity of light reflected at the recording layer.
